(19) United States
(12) Patent Application Publication
(10) Pub. No.: US 2013/0123572 A1
(43) Pub. Date: May 16, 2013

(54) SYSTEM AND METHOD FOR SOOTHING CHILDREN

(71) Applicant: Sigal ELIASI, Yavne (IL)

(72) Inventor: Sigal ELIASI, Yavne (IL)

(21) Appl. No.: 13/295,348

(22) Filed: Nov. 14, 2011

(51) Int. Cl. A61M 21/00

(52) U.S. Cl. 600/28

(57) ABSTRACT

The invention is a bracelet providing audio output adapted to soothe children. It provides a practical solution to the problem of babies who are fussy and having trouble sleeping. Whether the baby suffers from colic or a cold, is teething or just fussy, the inventive bracelet provides a possible solution to calming them down, soothing them enough to fall asleep.

### BACKGROUND

1. Technical Field

Embodiments of the present invention relate generally to systems and methods for a sound-producing bracelet.

2. Description of Related Art

Often children, toddlers and infants find themselves in states of emotional distress causing them to emit disturbing vocalizations. Generally these situations are dealt with by parents in a number of time consuming ways, for instance by providing care and attention to the child. However such steps as this involve an expenditure of time and effort that may well be spent elsewhere.

Hence, an improved method for soothing disturbed children is still a long felt need.

### BRIEF SUMMARY

It is within provision of the invention to disclose a soothing bracelet comprising:
a. a closed loop adapted to fit around the wrist; and,
b. sound producing means embedded within said loop;

whereby soothing sounds may be produced allowing a child to be lulled to sleep.

It is further within provision of the invention wherein said sound producing means comprise a power source, computing means, storage means, activation means, and playback means in electrical communication adapted to produce sound from digital representations thereof when activated by said activation means.

It is further within provision of the invention wherein said computing means are adapted to communicate with external agents for transfer of audio data to said storage means.

It is further within provision of the invention further comprising sensing means adapted for sensing conditions of child distress, said sensing means being in electronic communication with said computing means which are further adapted for producing sounds upon sensing child distress.

It is further within provision of the invention wherein said sensing means comprise a microphone and filter adapted for filtering frequencies characteristic of child distress.

It is further within provision of the invention further comprising sound selection means adapted for allowing the user to select from a variety of sounds for playback.

It is further within provision of the invention wherein said sounds are selected from the group consisting of: rhythmic drumming, bird songs, sundry woodland chirpings, rain, white noise, pink noise, wind chimes, vibraphone, ocean waves provide a sensation of relaxation and calm; ocean wave sounds, susurrant murmurings, river sounds, brook sounds, womb sounds, repetitive sounds.

It is within provision of the invention to disclose a method for soothing comprising steps of:
a. providing a closed loop adapted to fit around the wrist
b. providing sound producing means embedded within said loop;
c. activating said sound producing means;

whereby soothing sounds may be produced allowing a child to be lulled to sleep.

It is further within provision of the invention wherein said sound producing means comprise a power source, computing means, storage means, activation means, and playback means in electrical communication adapted to produce sound from digital representations thereof when activated by said activation means.

It is further within provision of the invention wherein said computing means are adapted to communicate with external agents for transfer of audio data to said storage means.

It is further within provision of the invention wherein said computing means are adapted to communicate with external agents for transfer of audio data to said storage means.

It is further within provision of the invention further comprising sensing means adapted for sensing conditions of child distress, said sensing means being in electronic communication with said computing means which are further adapted for producing sounds upon sensing child distress.

It is further within provision of the invention wherein said sensing means comprise a microphone and filter adapted for filtering frequencies characteristic of child distress.

It is further within provision of the invention further comprising sound selection means adapted for allowing the user to select from a variety of sounds for playback.

It is further within provision of the invention wherein said sounds are selected from the group consisting of: rhythmic drumming, bird songs, sundry woodland chirpings, rain, white noise, pink noise, wind chimes, vibraphone, ocean waves provide a sensation of relaxation and calm; ocean wave sounds, susurrant murmurings, river sounds, brook sounds, womb sounds, repetitive sounds.

These, additional, and/or other aspects and/or advantages of the present invention are: set forth in the detailed description which follows; possibly inferable from the detailed description; and/or learnable by practice of the present invention.

### BRIEF DESCRIPTION OF THE DRAWINGS

In order to understand the invention and to see how it may be implemented in practice, a plurality of embodiments will now be described, by way of non-limiting example only, with reference to the accompanying drawings, in which:

FIG. 1 illustrates a bracelet of the invention;

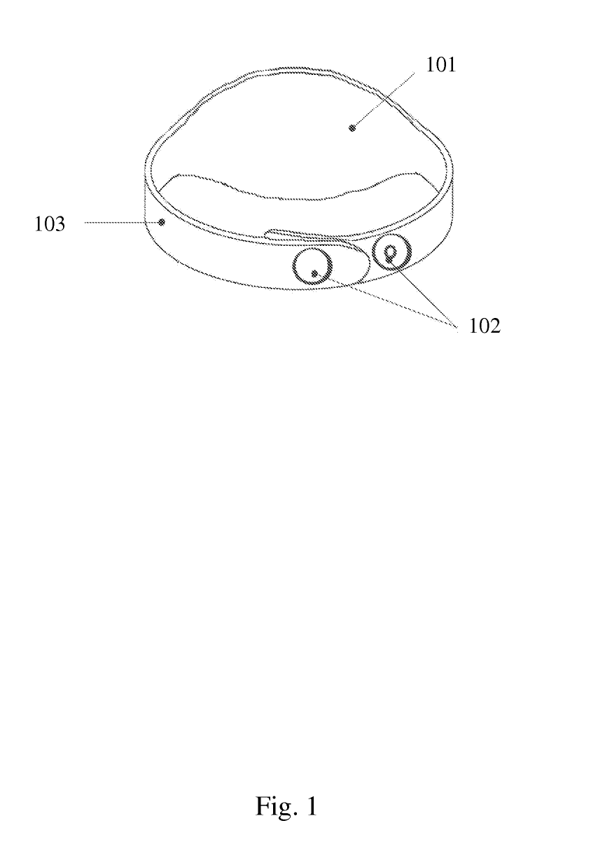

FIG. 2 illustrates another view of a bracelet consistent with one embodiment of the invention; and,

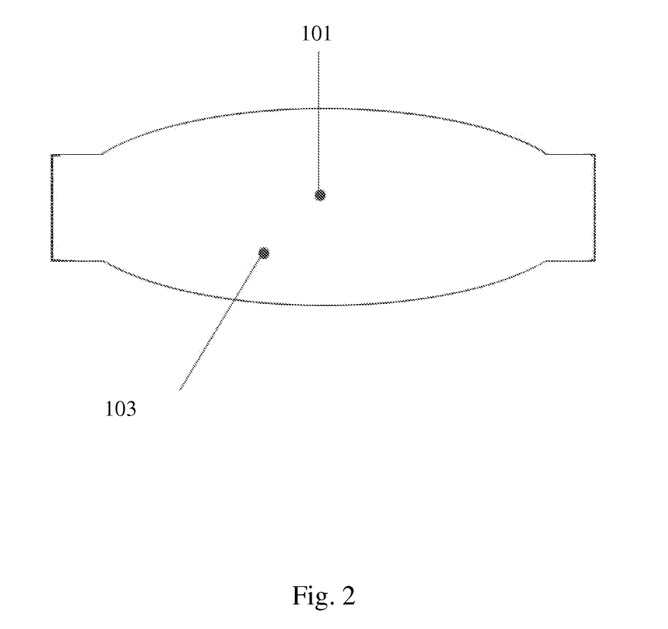

FIG. 3 illustrates yet another view of a bracelet consistent with one embodiment of the invention.

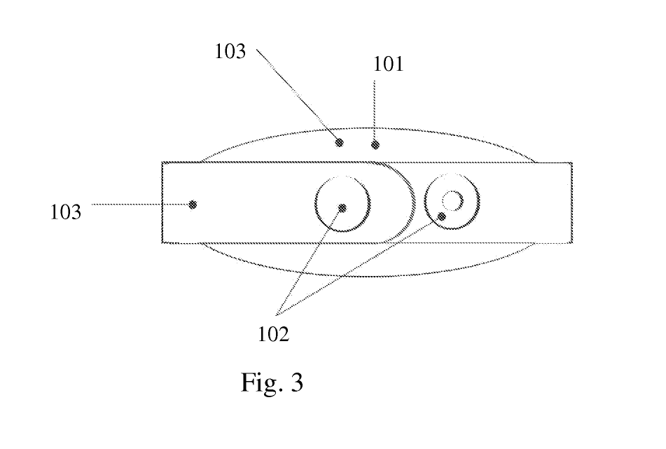

### DETAILED DESCRIPTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method for providing a system and method for a sound-producing bracelet.

In the following detailed description, numerous specific details are set forth in order to provide a thorough understanding of embodiments of the present invention. However, those skilled in the art will understand that such embodiments may be practiced without these specific details. Reference throughout this specification to “one embodiment” or “an embodiment” means that a particular feature, structure, or characteristic described in connection with the embodiment is included in at least one embodiment of the invention.

The term ‘plurality’ refers hereinafter to any positive integer (e.g. 1,5, or 10).

The invention may be described as a bracelet providing audio output adapted to soothe the swaddling child. It provides a practical solution to the problem of babies who are fussy and having trouble sleeping. Often a baby or toddler has trouble falling asleep, in some cases insisting on being held or rocked through the night. Whether the baby suffers from colic or a cold, is teething or just fussy, the inventive bracelet provides a possible solution to calming them down, soothing them enough to fall asleep.

There are few situations a human being can encounter in a normal lifetime more stressful than being confronted with a shrieking, colicky infant. The interminable bawling, the maddening wails, the cries of despair like missiles of hopelessness sent into the heart of an uncaring universe, and the discomfort and agony that one's baby goes through is only made worse by the helpless feeling that nothing one does seems to make it better.

But a shining beacon of hope has appeared over the dismal horizon! Forsooth, white noise has been proven to help relieve colic symptoms. Infant research and anecdotal experience alike have unflinchingly shown that a variety of sounds are all of effect to help a colicky infant relax and achieve the deepest slumbers.

Now comes the inventive bracelet, designed with built-in sounds adapted specifically for soothing children; for example sounds of rain and the sound of ocean waves.

The bracelet can provide an answer to a child's—and parents—sleepless nights. The utility of the device revolves around the need to provide quality portable bracelets, wearable by adults and/or children, that will help calm children at any given place or time. When a child hears the comforting sounds of the ocean gently lapping against the shore or a soft pattering of rain, a wave of comfort will wash over them and they will drift into an illimitable ocean of sleep.

The benefits of a well-rested child make it well worth using such a baby sound bracelet.

White noise and soothing sound create an environment that represents a safe place to your infant. When a child hears the comforting sounds that may be provided by the device, including for instance sounds of white noise, pink noise, rain, whale songs, ocean waves, and the like, they will drift to sleep due to the soothing soundscape immersing them and mentally transporting them to the endless sleep of the womb.

It is within provision of the invention to use a number of different sounds, including but not limited to the items in the following list:
a. Rhythmic drumming, which creates a trancelike hypnotic state to help induce sleep;
b. Bird songs and sundry woodland chirpings, which provide a light and airy sound-mosphere;
c. Rain, which can act as a pseudo-white noise machine;
d. Wind chimes, which provide ‘comforting’ sounds that provide a relaxing and stress free atmosphere;
e. The sonic output of the vibraphone may be calming;
f. The ancient sound of steady, rhythmic ocean waves provide a sensation of relaxation and calm, and which may remind one of the floating slumber of the womb to which we ultimately aspire.

As will be appreciated by one skilled in the art any of the above sonic landscapes may additionally masks household noises such as refrigerators, heaters, creaking floors, and the creeping footsteps of lurking burglars that may prove potentially terrifying to the keen imagination of the child.

It is within provision of the invention that it comprise 100% high quality silicone rubber, and in some embodiments may include the following components:
Sound playback device
Speaker
Noise sensor (for sensing the sound of a crying baby then activating the playback device and stopping it in some time interval after the baby has stopped crying)
On/off button
buttons (that will allow adjustability and portability)

It is within provision of the invention that the inventive bracelet comprise 100% high quality silicone rubber and certain embodiments may also include buttons. The complete series of bracelets, it is envisioned, will include a plurality of different soothing water-based sounds in a plurality of different colors and a plurality of different names, for instance:
Ocean Waves bracelet in blue
Mountain Lake bracelet in brown
River bracelet in green
Rainforest in light blue
Winter Winds in white

The inventive bracelet can be placed on an adult's hand and/or on an object such as stroller, crib, chair, toys, handbags and even on a seat belts of a car.

Although selected embodiments of the present invention have been shown and described, it is to be understood the present invention is not limited to the described embodiments. Instead, it is to be appreciated that changes may be made to these embodiments without departing from the principles and spirit of the invention, the scope of which is defined by the claims and the equivalents thereof.

What is claimed is:

1. A soothing bracelet comprising:
a. a closed loop adapted to fit around the wrist
b. sound producing means embedded within said loop;
whereby soothing sounds may be produced allowing a child to be lulled to sleep.

2. The device of claim 1 wherein said sound producing means comprise a power source, computing means, storage means, activation means, and playback means in electrical communication adapted to produce sound from digital representations thereof when activated by said activation means.

3. The device of claim 2 wherein said computing means are adapted to communicate with external agents for transfer of audio data to said storage means.

4. The device of claim 2 wherein said computing means are adapted to communicate with external agents for transfer of audio data to said storage means.

5. The device of claim 2 further comprising sensing means adapted for sensing conditions of child distress, said sensing means being in electronic communication with said computing means which are further adapted for producing sounds upon sensing child distress.

6. The device of claim 5 wherein said sensing means comprise a microphone and filter adapted for filtering frequencies characteristic of child distress.

7. The device of claim 2 further comprising sound selection means adapted for allowing the user to select from a variety of sounds for playback.

8. The device of claim 1 wherein said sounds are selected from the group consisting of: rhythmic drumming, bird songs, sundry woodland chirpings, rain, white noise, pink noise, wind chimes, vibraphone, ocean waves provide a sensation of relaxation and calm; ocean wave sounds, susurrant murmurings, river sounds, brook sounds, womb sounds, repetitive sounds.

9. A method for soothing comprising steps of:
a. providing a closed loop adapted to fit around the wrist
b. providing sound producing means embedded within said loop;
c. activating said sound producing means;
whereby soothing sounds may be produced allowing a child to be lulled to sleep.

10. The method of claim 9 wherein said sound producing means comprise a power source, computing means, storage means, activation means, and playback means in electrical communication adapted to produce sound from digital representations thereof when activated by said activation means.

11. The method of claim 9 wherein said computing means are adapted to communicate with external agents for transfer of audio data to said storage means.

12. The method of claim 9 wherein said computing means are adapted to communicate with external agents for transfer of audio data to said storage means.

13. The method of claim 9 further comprising sensing means adapted for sensing conditions of child distress, said sensing means being in electronic communication with said computing means which are further adapted for producing sounds upon sensing child distress.

14. The method of claim 13 wherein said sensing means comprise a microphone and filter adapted for filtering frequencies characteristic of child distress.

15. The method of claim 9 further comprising sound selection means adapted for allowing the user to select from a variety of sounds for playback.

16. The method of claim 15 wherein said sounds are selected from the group consisting of: rhythmic drumming, bird songs, sundry woodland chirpings, rain, white noise, pink noise, wind chimes, vibraphone, ocean waves provide a sensation of relaxation and calm; ocean wave sounds, susurrant murmurings, river sounds, brook sounds, womb sounds, repetitive sounds.
